(19) United States
(12) Patent Application Publication
(10) Pub. No.: US 2023/0234103 A1
(43) Pub. Date: Jul. 27, 2023

(54) E-Duster Device

(71) Applicant: Dima Hnoosh, Ownings Mills, MD (US)

(72) Inventor: Dima Hnoosh, Ownings Mills, MD (US)

(21) Appl. No.: 17/826,306

(22) Filed: May 27, 2022

Related U.S. Application Data
(60) Provisional application No. 63/301,646, filed on Jan. 21, 2022.

(51) Int. Cl. B08B 1/00; B08B 11/00

(52) U.S. Cl. CPC B08B 1/006; B08B 11/00; A61B 90/70

(57) ABSTRACT

The present invention relates to a novel miniature duster device designed to provide users with a device capable of cleaning delicate medical equipment, small electronics and other fragile items. The device preferably comprises a cylindrical body with two opposite telescoping ends. One end is comprised of a microfiber dusting component, while the opposite end is comprised of a feather dusting component. Users can selectively expand one or both ends and apply them to the object for simple and convenient cleaning. Further, the miniature duster device comprises an attachment point for a key ring for easy transportation.

### CROSS-REFERENCE TO RELATED APPLICATION

The present application claims priority to, and the benefit of, U.S. Provisional Application No. 63/301,646, which was filed on Jan. 21, 2022 and is incorporated herein by reference in its entirety.

### FIELD OF THE INVENTION

The present invention relates generally to the field of dusters. More specifically, the present invention relates to an improved miniature duster device for cleaning delicate medical equipment, small electronics, and other fragile items. The device preferably comprises a cylindrical or other shaped body with at least one and preferably opposing telescoping ends, which includes a microfiber dusting end and a feather dusting end or other accessory. Accordingly, the present disclosure makes specific reference thereto. Nonetheless, it is to be appreciated that aspects of the present invention are also equally applicable to other like applications, devices and methods of manufacture.

### BACKGROUND

By way of background, dusting involves the employment of a dusting device, such as a feather duster, intended for removing dust from surfaces and between tight spaces. However, as a user passes conventional dusting devices over a surface or area containing dust, especially delicate equipment and keyboards, the dust particles are simply moved around or disturbed and released into the air, only to settle back onto a surface moments later. As such, dust continues to remain in the home where it can cause health issues, contain dust mites, clog and potentially harm electronic devices.

Furthermore, cleaning delicate medical equipment, keyboards, computer towers, and other types of electronics with fragile and sensitive parts can be difficult. Standard vacuums and hoses can be dangerous to use and have too strong of suction. Additionally, using these types of vacuums on sensitive items can cause parts to come loose and be suctioned into the vacuum. However, without cleaning delicate medical equipment, keyboards and other electronics, they can become dirty, grimy, and unpleasant to use.

Therefore, hard to reach areas such as high places and between small spaces continue to go uncleaned. As such, there is a continuing unmet need for a cleaning device which provides a means to completely and effectively remove dust. Therefore, users require a device that easily removes dust and dirt from delicate medical equipment, small electronics, and other fragile items.

Therefore, there exists a long-felt need in the art for a miniature duster device that provides users with a device capable of cleaning delicate medical equipment, small electronics and other fragile items. There is also a long-felt need in the art for a miniature duster device that prevents damage to expensive and delicate items that need to be cleaned. Further, there is a long-felt need in the art for a miniature duster device that features an expanding and retracting microfiber duster that can be selectively used on different types of objects. Moreover, there is a long-felt need in the art for a device that includes a second feather duster on the opposite end to further clean items and objects. Further, there is a long-felt need in the art for a miniature duster device that comprises an attachment point for a keychain, allowing users to easily transport the duster device and constantly keep it on hand. Finally, there is a long-felt need in the art for a miniature duster device that provides users with a unique and convenient device for cleaning delicate objects.

The subject matter disclosed and claimed herein, in one embodiment thereof, comprises a miniature duster device. The device is designed to provide users with a device capable of cleaning delicate medical equipment, small electronics and other fragile items. The device comprises a cylindrical body with two opposite telescoping ends. One end is comprised of a microfiber dusting component, while the opposite end is comprised of a feather dusting component. Users can selectively expand one or both ends and apply them to the object for simple and convenient cleaning. Further, the miniature duster device comprises an attachment point for a key ring for easy transportation, or a key ring or magnet itself.

In this manner, the miniature duster device of the present invention accomplishes all of the foregoing objectives and provides users with a device that conveniently cleans delicate medical equipment, small electronics, and other fragile items. The device prevents damage to expensive and delicate items that need to be cleaned. The device allows users to selectively expand one or both ends of the device and apply them to the object for simple and convenient cleaning.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a miniature duster device. The device is designed to provide users with a device capable of cleaning delicate medical equipment, small electronics and other fragile items. The device comprises a cylindrical body with at least one and preferably two opposite telescoping ends. One end is comprised of a microfiber dusting component, while the opposite end is comprised of a feather dusting component. Alternatively, other accessories may be positioned on the ends such as, but not limited to, a torch, blank, etc. Users can selectively expand one or both ends and apply them to the object for simple and convenient cleaning.

In yet another embodiment, the cylindrical body of the miniature duster device is manufactured from a wood or a plastic or polymers, such as polypropylene or acrylonitrile-butadiene-styrene (ABS), or metals, such as aluminum or stainless steel, etc.

In one embodiment, the cylindrical body of the miniature duster device is hollow allowing the microfiber dusting component and the feather dusting component to be stored within the body when not in use. In another embodiment, the cylindrical body can be partially solid, as long as there is room for the microfiber and feather dusting component to be stored within.

In one embodiment, the telescoping ends utilize a sliding actuator, a spring-loaded release, a twist lock, etc., or any other telescoping mechanism as is known in the art to expand or retract the two ends.

In one embodiment, the miniature duster device comprises a microfiber dusting component which comprises a rod component secured within the cylindrical body and microfiber material secured at the upper end of the rod component. The microfiber material comprises light filaments and/or plastic filaments that is usually used to remove or sweep dust which is present on delicate medical equipment, electronic products, etc. The lower end of the rod component includes any suitable attachment means, which secures the rod component within the cylindrical hollow body.

In one embodiment, the miniature duster device comprises a feather dusting component which comprises a rod component secured within the cylindrical body and feathers secured at the end of the rod component. The feathers comprise an outer concentric ring of ostrich feathers enclosing an inner concentric ring of turkey feathers, which encircle an inner concentric center ring portion of ostrich feathers all secured by a suitable fastening means to or near the upper end of the rod component. The lower end of the rod component includes any suitable attachment means, which secures the rod component within the cylindrical hollow body.

In yet another embodiment, the miniature duster device comprises an attachment point for a key ring for easy transportation. The attachment point is typically a ring or metal loop secured to the cylindrical body for retaining a key ring.

In another embodiment, the miniature duster device comprises a plurality of indicia on the cylindrical body.

In yet another embodiment, a method of cleaning delicate medical equipment, keyboards, and other electronics is described. The method includes the steps of providing a cylindrical body with two opposite telescoping ends, wherein one end is comprised of a microfiber dusting component, while the opposite end is comprised of a feather dusting component. The method also comprises expanding one or both of the ends for use. Further, the method comprises applying the expanded ends to the object to be cleaned. Finally, retracting the telescoping ends for storage. Further, the method can comprise securing the cylindrical body to a keychain for easy transportation.

In yet another embodiment of the present invention, a miniature duster device is disclosed, which provides users with a device capable of cleaning delicate medical equipment, small electronics and other fragile items. The device comprises a cylindrical body with two opposite telescoping ends. One end is comprised of a microfiber dusting component, while the opposite end is comprised of a feather dusting component. Users can selectively expand one or both ends and apply them to the object for simple and convenient cleaning. Further, the miniature duster device comprises an attachment point for a key ring for easy transportation.

Numerous benefits and advantages of this invention will become apparent to those skilled in the art to which it pertains, upon reading and understanding the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of the miniature duster device of the present invention in an extended position in accordance with the disclosed architecture;

FIG. 2 illustrates a perspective view of another potential embodiment of the miniature duster device of the present invention in a compact position in accordance with the disclosed architecture;

FIG. 3 illustrates a perspective view of another potential embodiment of the miniature duster device of the present invention in use to clean a computer keyboard in accordance with the disclosed architecture;

FIG. 4 illustrates a perspective view of another potential embodiment of the miniature duster device of the present invention in use to clean a smart phone in accordance with the disclosed architecture;

FIG. 5 illustrates a perspective view of one potential embodiment of the miniature duster device of the present invention attached to a keychain in accordance with the disclosed architecture; and

FIG. 6 illustrates a flowchart showing the method of cleaning delicate medical equipment, keyboards, and other electronics using the miniature duster device of the present invention in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for a miniature duster device that provides users with a device capable of cleaning delicate medical equipment, small electronics and other fragile items. There is also a long-felt need in the art for a miniature duster device that prevents damage to expensive and delicate items that need to be cleaned. Further, there is a long-felt need in the art for a miniature duster device that features an expanding and retracting microfiber duster that can be selectively used on different types of objects. Moreover, there is a long-felt need in the art for a device that includes a second feather duster on the opposite end to further clean items and objects. Further, there is a long-felt need in the art for a miniature duster device that comprises an attachment point for a keychain, allowing users to easily transport the duster device and constantly keep it on hand.

The present invention, in one exemplary embodiment, is a novel miniature duster device comprising a cylindrical body with at least one, and preferably two opposite telescoping ends. One end is comprised of a microfiber dusting component or other accessory or no accessory, while the opposite end is comprised of a feather dusting component or other accessory or no accessory. Users can selectively expand one or both ends and apply them to the object for simple and convenient cleaning. The present invention also includes a novel method of cleaning delicate medical equipment, small electronics and other fragile items. The method includes the steps of providing a cylindrical body with two opposite telescoping ends. The method also comprises expanding one or both of the ends for use. Further, the method comprises applying the expanded ends to the object to be cleaned. Finally, retracting the telescoping ends for storage.

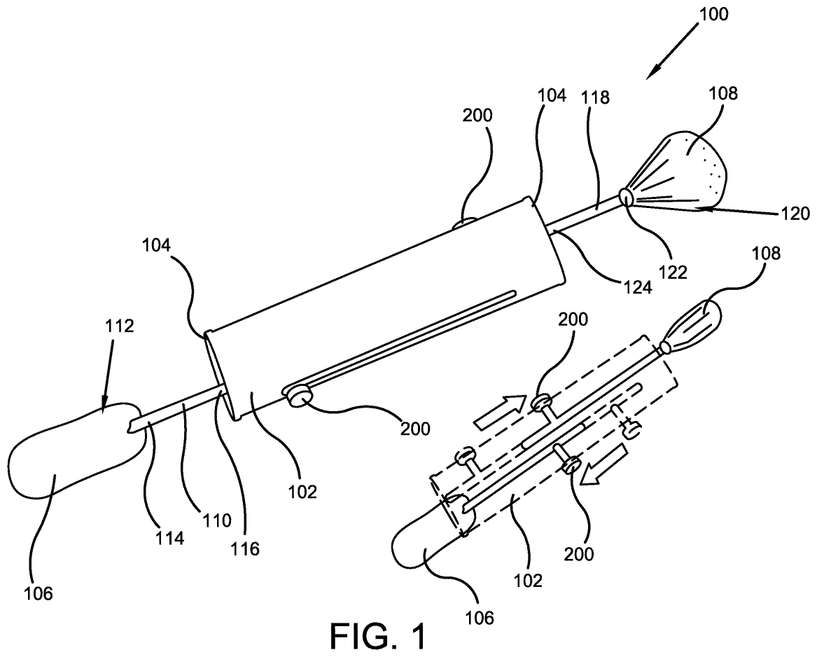

Referring initially to the drawings, FIG. 1 illustrates a perspective view of one embodiment of the miniature duster device 100 of the present invention. In the present embodiment, the miniature duster device 100 is an improved dusting device that provides a simple and convenient way to clean electronic equipment and other delicate items. The device 100 is especially designed to allow any suitable user, as is known in the art, to easily clean electronics and other delicate equipment. More specifically, the device 100 comprises a cylindrical body 102 with two opposite telescoping ends 104. Typically, the body 102 is cylindrical, but can be any suitable shape as is known in the art.

Further, one end is comprised of a microfiber dusting component 106, while the opposite end is comprised of a feather dusting component 108. Users can selectively expand one or both ends 106 and 108 and apply them to the object for simple and convenient cleaning. Any suitable dusting components as is known in the art can be utilized on either end of the cylindrical body 102, depending on the wants and/or needs of a user. Further, both ends do not need to comprise a dusting component, only one end can comprise a dusting component and the other end is solid, depending on the wants and/or needs of a user.

In one embodiment, the microfiber dusting component 106 comprises a rod component 110 secured within the cylindrical body 102 and microfiber material 112 secured at the upper end 114 of the rod component 110. The microfiber material 112 comprises light filaments and/or plastic filaments, etc., or any other suitable material as is known in the art, it is usually used to remove or sweep dust which is present on delicate medical equipment, electronic products, etc. The lower end 116 of the rod component 110 includes any suitable attachment means, which secures the rod component 110 within the cylindrical hollow body 102.

In another embodiment, the feather dusting component 108 comprises a rod component 118 secured within the cylindrical body 102 and feathers 120 secured at the upper end 122 of the rod component 118. The feathers 120 comprise an outer concentric ring of ostrich feathers enclosing an inner concentric ring of turkey feathers, which encircle an inner concentric center ring portion of ostrich feathers all secured by suitable fastening means to or near the upper end 122 of the rod component 118. Notwithstanding, other types of dusting materials may also be used, such as fabrics. The lower end 124 of the rod component 118 includes any suitable attachment means, which secures the rod component 118 within the cylindrical hollow body 102.

In another embodiment, the cylindrical body 102 of the miniature duster device 100 is manufactured from a wood, plastic or polymer, such as polypropylene or acrylonitrile-butadiene-styrene (ABS), acrylic, polycarbonate, polyethylene, polyvinyl chloride, polystyrene, or metals, such as aluminum or stainless steel, etc., or any other suitable material as is known in the art.

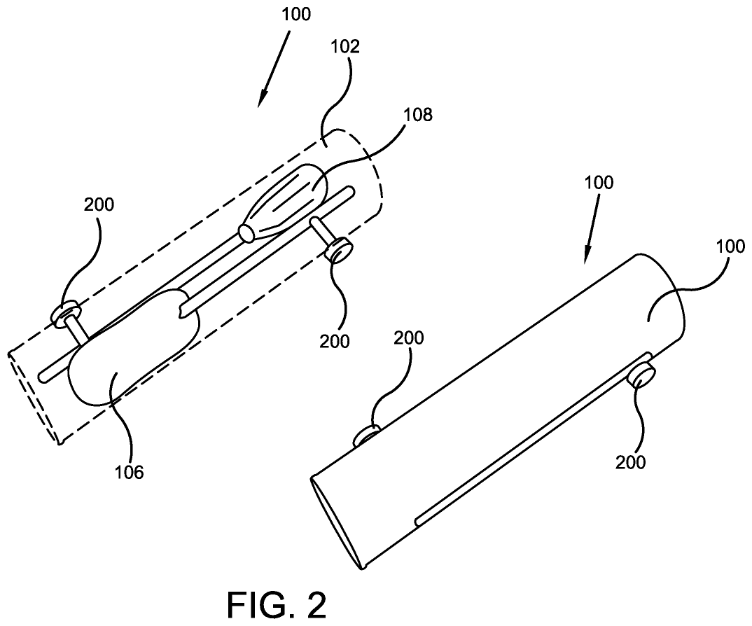

FIG. 2 illustrates a perspective view of another embodiment of the miniature duster device 100 of the present invention. In this embodiment, the cylindrical body 102 of the miniature duster device 100 is hollow allowing the microfiber dusting component 106 and the feather dusting component 108 to be stored within the body 102 when not in use. In another embodiment, the cylindrical body 102 can be partially solid, as long as there is room for the microfiber 106 and feather dusting component 108 to be stored within.

Further, the telescoping ends 104 of the miniature duster device 100 utilize a sliding actuator 200, a spring-loaded release, a twist lock, etc., or any other telescoping mechanism as is known in the art to expand or retract the two ends 104 to expose either the microfiber dusting component 106 or the feather dusting component 108, or both.

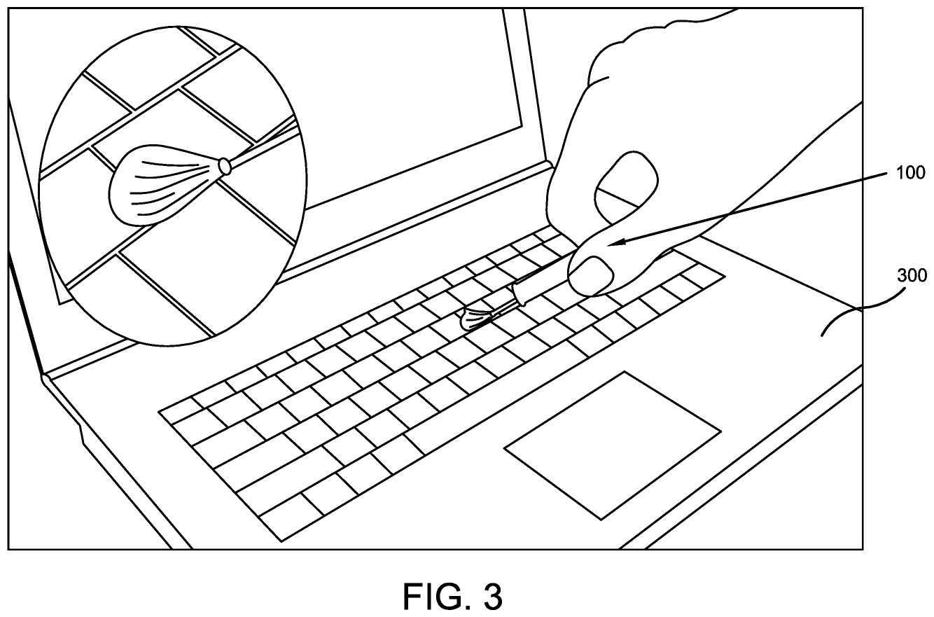

FIG. 3 illustrates a perspective view of another embodiment of the miniature duster device 100 of the present invention in use. In this embodiment, the miniature duster device 100 allows users to clean delicate medical equipment, small electronics 300 and other fragile items. The device 100 comprises a cylindrical body 102 with two opposite telescoping ends 104. One end is comprised of a microfiber dusting component 106, while the opposite end is comprised of a feather dusting component 108. Users can selectively expand one or both ends 104 and apply them to the electronics 300, or other suitable items as is known in the art, for simple and convenient cleaning.

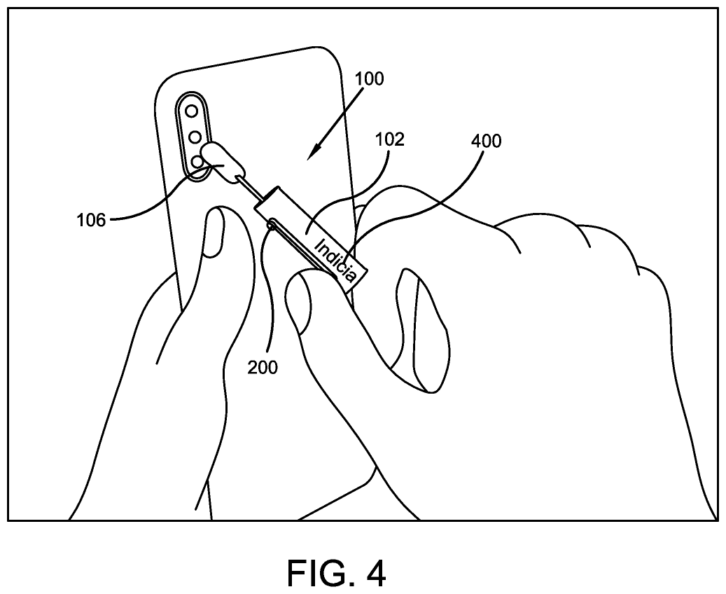

FIG. 4 illustrates a perspective view of another embodiment of the miniature duster device 100 of the present invention in use. In this embodiment, the miniature duster device 100 comprises a plurality of indicia 400 on the cylindrical body 102. The cylindrical body 102 may include advertising, a trademark, or other letters, designs, or characters, printed, painted, stamped, or integrated into the body 102, or any other indicia 400 as is known in the art. Specifically, any suitable 400 indicia as is known in the art can be included, such as, but not limited to, patterns, logos, emblems, images, symbols, designs, letters, words, characters, animals, advertisements, brands, etc., that may or may not be dusting/electronics related.

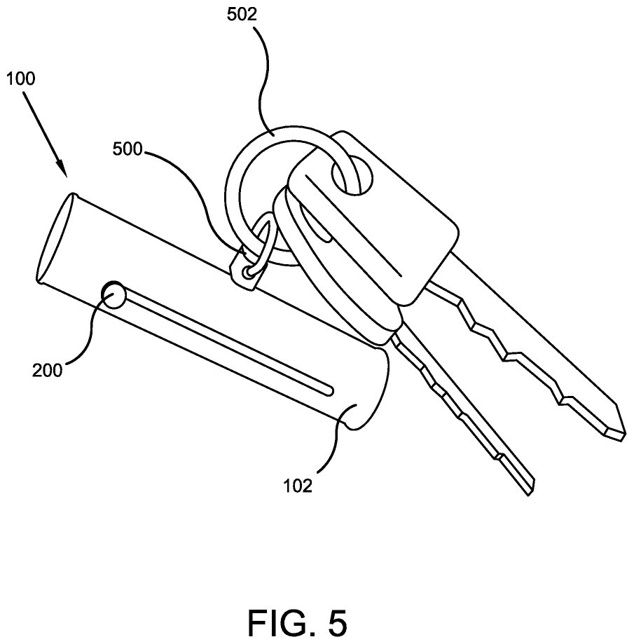

FIG. 5 illustrates a perspective view of another embodiment of the miniature duster device 100 of the present invention. In this embodiment, the miniature duster device 100 comprises an attachment point 500 for a key ring 502 for easy transportation, or a key ring 502 or magnet itself. The attachment point 500 is typically a ring or metal loop secured to the cylindrical body 102 for retaining a key ring 502, but can be any suitable attachment means as is known in the art. The miniature duster device 100 can also be used for other purposes, such as cleaning and dusting small decorations, like crystals, ornament, jewelry, etc.

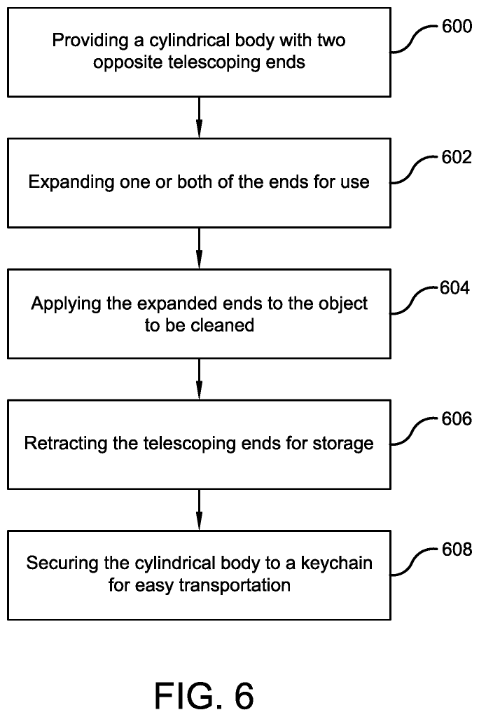

FIG. 6 illustrates a flowchart of the method of cleaning delicate medical equipment, keyboards, and other electronics. The method includes the steps of at 600, providing a cylindrical body with two opposite telescoping ends, wherein one end is comprised of a microfiber dusting component, while the opposite end is comprised of a feather dusting component. The method also comprises at 602, expanding one or both of the ends for use. Further, the method comprises at 604, applying the expanded ends to the object to be cleaned. Finally, at 606, retracting the telescoping ends for storage. Further, the method can comprise at 608, securing the cylindrical body to a keychain for easy transportation.

Certain terms are used throughout the following description and claims to refer to particular features or components. As one skilled in the art will appreciate, different users may refer to the same feature or component by different names. This document does not intend to distinguish between components or features that differ in name but not structure or function. As used herein “miniature duster device” and “device” are interchangeable and refer to the miniature duster device 100 of the present invention. Further, the body is not limited to a cylindrical shape and can be any shape that satisfied user need and/or preference.

Notwithstanding the foregoing, the miniature duster device 100 of the present invention can be of any suitable size and configuration as is known in the art without affecting the overall concept of the invention, provided that it accomplishes the above-stated objectives. One of ordinary skill in the art will appreciate that the miniature duster device 100 as shown in FIGS. 1-6 is for illustrative purposes only, and that many other sizes and shapes of the miniature duster device 100 are well within the scope of the present disclosure. Although the dimensions of the miniature duster device 100 are important design parameters for user convenience, the miniature duster device 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A miniature duster device for providing a simple and convenient way to clean an electronic device and other delicate items, the miniature duster device comprising:
a cylindrical body having a pair of opposite telescoping ends;
a microfiber dusting component positioned on a first of the pair of opposite telescoping ends; and
a feather dusting component positioned on a second of the pair of opposite telescoping ends, wherein a user may selectively expand one or both of the pair of opposite telescoping ends to apply the microfiber dusting component or the feather dusting component to an object for cleaning.

2. The miniature duster device of claim 1 further comprising a rod component secured within the cylindrical body and a microfiber material secured to an end of the rod component.

3. The miniature duster device of claim 2, wherein the microfiber material comprises a plurality of light filaments or a plurality of plastic filaments.

4. The miniature duster device of claim 3, wherein the feather dusting component comprises a rod component secured within the cylindrical body and a plurality of feathers secured to an end of the rod component.

5. The miniature duster device of claim 4, wherein the plurality of feathers comprise a plurality of ostrich feathers or a plurality of turkey feathers.

6. The miniature duster device of claim 1, wherein the cylindrical body is hollow.

7. The miniature duster device of claim 1, wherein the cylindrical body comprises a sliding actuator to expand or retract at least one of the pair of opposite telescoping ends.

8. The miniature duster device of claim 1, wherein the cylindrical body comprises a plurality of indicia.

9. The miniature duster device of claim 1, wherein the cylindrical body comprises an attachment point for a key ring.

10. The miniature duster device of claim 9, wherein the attachment point comprises a metal ring secured to the cylindrical body.

11. The miniature duster device of claim 1, wherein the miniature duster device is manufactured from at least one of a polypropylene, an acrylonitrile-butadiene-styrene (ABS), an acrylic, a polycarbonate, a polyethylene, a polyvinyl chloride, a polystyrene, an aluminum or a stainless steel.

12. A miniature duster device for providing a simple and convenient way to clean an electronic device or other delicate item, the miniature duster device comprising:
a hollow body having at least one telescoping end;
a sliding actuator to expand or retract the at least one telescoping end; and
a dusting component comprising a rod component secured within the hollow body and a material secured to an end of the rod component of the dusting component, wherein a user can selectively expand the at least one telescoping end.

13. The miniature duster device of claim 12, wherein the hollow body comprises a plurality of indicia.

14. The miniature duster device of claim 12, wherein the miniature duster device is manufactured from at least one of a wood, a polypropylene, an acrylonitrile-butadiene-styrene (ABS), an acrylic, a polycarbonate, a polyethylene, a polyvinyl chloride, a polystyrene, an aluminum or a stainless steel.

15. The miniature duster device of claim 12, wherein the hollow body comprises an attachment point for a key ring, the key ring or a magnet.

16. The miniature duster device of claim 15, wherein the attachment point comprises a metal ring secured to the hollow body.

17. The miniature duster device of claim 12, wherein the material comprises a plurality of light filaments, a plurality of plastic filaments, a cloth or a plurality of feathers secured to an end of the rod component of the dusting component.

18. The miniature duster device of claim 17, wherein the plurality of feathers comprise a plurality of ostrich feathers and a plurality of turkey feathers.

19. A method of using a device to clean delicate medical equipment, keyboards and other electronics, the method comprising the steps of:
providing the device having a cylindrical body with a pair of opposite telescoping ends, wherein a first of the pair of opposite telescoping ends is comprised of a microfiber dusting component, and a second of the pair of opposite telescoping ends is comprised of a feather dusting component;
expanding at least one of the first and second of the pair of opposite telescoping ends for use;
applying the expanded ends to an object to be cleaned; and
retracting the two opposite telescoping ends for storage.

20. The method of claim 19 further comprising a step of securing the cylindrical body to a keychain for easy transportation.
